(19) United States
(12) Patent Application Publication
(10) Pub. No.: US 2020/0102529 A1
(43) Pub. Date: Apr. 2, 2020

(54) EQUIPMENT AND METHOD FOR ADDITIVE MANUFACTURING

(71) Applicant: Poietis, Pessac (FR)

(72) Inventors: Fabien Guillemot, Preignac (FR); Bertrand Viellerobe, Merignac (FR); Jérôme Bouter, Bordeaux (FR); Evarzeg Le Bouffant, Merignac (FR); Romain Voucelle, Bordeaux (FR); Dan Soto, Bordeaux (FR)

(21) Appl. No.: 16/494,737

(22) PCT Filed: Mar. 8, 2018
(86) PCT No.: PCT/FR2018/050536

§ 371 Date: Sep. 16, 2019

(30) Foreign Application Priority Data
Mar. 15, 2017 (FR) 1752131

(51) Int. Cl. C12M 3/00; A61L 27/38; B33Y 10/00; B33Y 30/00

(52) U.S. Cl. CPC C12M 21/08; B33Y 30/00; B33Y 10/00; A61L 27/38

(57) ABSTRACT

The present disclosure relates to equipment and a method for additive manufacturing, comprising an orientable energy excitation means for generating intermittent interaction with a fluid covering a blade in order to trigger a jet oriented in the direction of a target, the fluid consisting of a liquid vector containing inhomogeneities, wherein: the fluid forms a liquid film of a thickness measuring less than 500 μm on a blade having at least one area allowing the interaction with the laser, into which at least one inlet leads, the interaction area leading into at least one outlet, the equipment also comprising means for the circulation of the fluid between the inlet and the outlet.

### CROSS-REFERENCE TO RELATED APPLICATIONS

This application is a national phase entry under 35 U.S.C. § 371 of International Patent Application PCT/FR2018/050536, filed Mar. 8, 2018, designating the United States of America and published as International Patent Publication WO 2018/167402 A1 on Sep. 20, 2018, which claims the benefit under Article 8 of the Patent Cooperation Treaty to French Patent Application Serial No. 1752131, filed Mar. 15, 2017.

### TECHNICAL FIELD

The present disclosure concerns the field of three-dimensional additive laser-assisted printing and more particularly, but not exclusively, bio-printing.

### BACKGROUND

Laser-assisted three-dimensional additive printing consists of projecting particles toward a target by a jet produced by the local vaporization of a substrate providing kinetic energy to a transferable particle or aggregate of particles contained in a carrier fluid.

Transferable particles can come from a powdery material carried on a liquid substrate, or a liquid transferable material.

They can be metallic or inorganic compounds, polymers or biomaterials.

They can also be made up of biological particles, for example, living cells.

The present disclosure concerns the field of laser bio-printing by a computer-assisted transfer process for modelling and assembling living and optionally non-living materials with a prescribed 2D or 3D organization in order to produce bioengineered structures for use in regenerative medicine, pharmacology and cell biology studies.

Tissue engineering aims to design and develop biologically suitable alternatives to replace, restore or maintain the functions of native tissue or even an organ. An example is described in the article by Griffith, L. G., & Naughton, G., Tissue engineering—current challenges and expanding opportunities, Science, 295(5557), 1009-1014 (2002).

To overcome these limitations, the printing of biological elements, more commonly referred to as bio-printing, began to be imagined, as discussed in Klebe, R. Cytoscribing: A Method for Micropositioning Cells and the Construction of Two- and Three-Dimensional Synthetic Tissues, Experimental Cell Research, 179(2):362-373 (1988), and in Klebe, R., Thomas, C., Grant, G. Grant, A. and Gosh, P., Cytoscription: Computer controlled micropositioning of cell adhesion proteins and cells, Methods in Cell Science, 16(3):189-192 (1994).

Patent Application Publication WO2016097619 describes a method and equipment for printing with at least one ink, the method comprising a step of focusing a laser beam so as to generate a cavity in an ink film, a step of forming at least one ink droplet from a free surface of the ink film and a step of depositing the droplet onto a depositing surface of a receiving substrate, characterized in that the laser beam is oriented in the direction opposite to the gravitational force, the free surface of the film being oriented upwards toward the depositing surface placed over the ink film.

Patent Application Publication WO2014061024 describes a system for laser-induced forward transfer (LIFT) without substrate and/or with a local donor. This system includes a tank with at least one opening. An energy source is configured to deliver energy to a donor material within the reservoir. This system allows the deposition of material by laser-induced forward transfer without the need for a donor substrate. The present disclosure also covers forward transfer processes induced by laser without substrate and with a local donor.

In the solutions of the prior art providing for a blade coated with a fluid containing the particles to be transferred, as it is necessary to replace the blade after each sequence, which does not allow for good control of the characteristics of the film containing the transferable elements, in particular, the volume of the fluid, the spreading of the fluid on the surface of the blade, the homogeneity and the development over time due to drying, evaporation, evolution of living particles, etc. Thus, the main disadvantage of prior solutions concerns both a lack of reproducibility of prints and the need for frequent handling, which reduce productivity.

In solutions involving a tank or reservoir containing a fluid, it is difficult to control the generation of the jet at the air-liquid interface, as the materials to be transferred tend to settle and thus to be far from the target. These solutions therefore do not allow the transfer of particles contained in a fluid under reproducible conditions. This is a generic disadvantage also known for bio-extrusion or ink-jet solutions.

In general, previously known solutions are not suitable for an industrial process for printing liquid media containing particles, due to the difficulty of accurately targeting the particles contained in the fluid and the need to change the substrate regularly. These changes in support require manipulations, resulting in the lack of reproducibility observed.

A secondary problem that the present disclosure aims to remedy by some of its variants concerns the abandonment of the interaction of the laser with a sacrificial layer, for example, a gold coating.

For the previously known solutions that use a sacrificial layer, the jet formed by a laser exciting this layer causes the transfer of material from this layer, which can lead to problems of toxicity and projection of particles other than the particles to be transferred.

The previously known solutions also involve the local destruction of the substrate during each laser pulse, which creates inhomogeneities and requires a repeated change of the substrate.

### BRIEF SUMMARY

Inhomogeneity of the bio-ink film within the meaning of this patent application means any area of the film with specific local characteristics in terms of composition: particles, biochemical species (growth factor, molecules, ions), biomaterials.

The terms “inhomogeneous zone”, “local variations in composition”, “zone of specific composition” have the same technical meaning within the meaning of this patent.

The solution consists in making the inhomogeneities of the fluid film positioned in the laser interaction zone homogeneous in thickness and volume density during the three-dimensional additive laser-assisted printing. It also consists in allowing the filling of the interaction zone in a repeated and controlled way by the fluid. Such a solution requires the implementation of a laser printing process that does not use a sacrificial layer for the generation of material jets, which effectively involves a laser-material interaction that takes place directly in the fluid. Thus, the process of generating the cavitation bubble and then the material jet will be different from the previously known techniques.

The advantages of this solution are manifold:
it allows to bring, in a controlled and reproducible way, the inhomogeneous fluid toward the interaction zone by avoiding any manipulation (pipetting, spreading, cleaning, etc.). It therefore makes the process more secure and reliable;
it allows the composition of the ink to be modulated by mixing several liquids (chemical species, liquid biomaterials, etc.) and several types of particles (cells, biomaterials, etc.) in the same type of ink;
it allows the use of a controllable fluidic system that continuously or pseudo-continuously recharges the interaction zone, thus increasing printing productivity;
it makes it possible to obtain a film of fluid homogeneous in thickness on the surface of the interaction zone, which aims to make the printing much more reproducible and homogeneous at the level of the droplets printed on the receiving surface;
optionally, the films can have a slope or shape appropriate to both the geometry of the inputs/outputs and to the filling mode (continuous, discontinuous, round trip, etc.); and
it allows the adjustment of the film thickness thanks to optimized parameter sets (flow rate, section, shape, etc.) of the fluid system. Thus, the height of the material jets can be adjusted in this way, which can be very interesting for printing on non-planar surfaces.

In addition, the present disclosure allows the use of imaging means correlated to laser pulses in order to target inhomogeneities in the fluid in a controlled manner. In order to achieve this, the interaction area must be based on a transparent material, both for the laser and for the image acquisition means.

Although this solution is compatible with laser printing based on the use of a sacrificial layer (typically a metallic layer of gold or silver), it is preferably intended for laser printing without the use of a sacrificial layer. Such a solution must therefore ensure the creation of reproducible and repeatable jets in the field during direct interaction between the laser and the fluid containing the inhomogeneities. In order to achieve this, a number of printing parameters, listed below, are necessary because jet generation is very difficult to achieve in this printing condition without a sacrificial layer:
the laser emits short pulses in picosecond or femtosecond mode with an energy level between 1 to 40 microjoules, and preferably 5 to 20 microjoules in order to optimize the generation of the laser plasma in the fluid. Upon reading the examples of embodiments that will be described in this document, proof of these performances will be provided;
the laser emits laser pulses in nanosecond mode with an energy of 0.5 to 20 millijoules to allow the generation of bubbles and then jets without sacrificial layer;
the laser preferably emits pulses in the near IR range to avoid any ionizing effect on the cells while at the same time being sufficiently absorbable by the medium. To optimize this last parameter, it would be quite possible to use a laser in the UV or medium IR, or even in the visible in order to maximize the rate of absorption by the medium;
measurements of the characteristic properties of the fluid present in the interaction zone are carried out (density, viscosity, film thickness, etc.) in order to modulate or optimize the laser parameters and make the printing as homogeneous as possible; and
the images of the inhomogeneities in the fluid make it possible to target specific areas (number or type of particles), which again makes it possible to make the printing homogeneous and above all compliant with the digital printing file since the number of inhomogeneities printed can be directly controlled by these imaging means or more generally by computer-controlled characterization for the interpretation of the acquired data.

In such a context, there is no longer any problem related to the printing of debris from the sacrificial layer to the printing substrate, thus ensuring a higher viability of the cells in the context of bio-printing.

The present disclosure concerns, in its most general sense, an additive printing equipment comprising a directable energy excitation means for producing a point interaction with a fluid covering a slide, in order to cause a jet oriented toward a target, the fluid being constituted by a liquid vector containing transferable particles or by a transferable liquid biomaterial, characterized in that:
the fluid forms a liquid film with a thickness of less than 500 m;
on a slide having at least one zone allowing interaction with the laser in which at least one inlet opens, the interaction zone opening into at least one outlet, the interaction zone having an opening whose cross-section is at least three times larger than the median size of the inhomogeneities present in the fluid; and
the equipment further comprising means for circulating the fluid between the inlet and the outlet.

This slide defines an area with a preferably flat bottom, positioned to allow interaction with the energy excitation beam, this area being surrounded by a border having an inlet and an outlet opening, to ensure the presence, in this interaction area with the energy excitation means, of a film that can be transiently static, deposited on the area, and at other times formed by a fluid circulation ensuring the renewal of transferable particles and displacement with respect to the energy excitation axis.

In “static” solutions, it is necessary to replace the slide after each sequence. This does not allow a good control of the characteristics of the film containing the transferable elements, in particular, the volume of the fluid, the spreading of the fluid on the surface of the slide, the homogeneity and the development over time due to the phenomena of drying, evaporation, the evolution of living particles, etc.

The slides of the prior art have a static coating of an ink, requiring the slide to be changed after each use, which does not optimize the use of transferable elements.

According to specific embodiments of the equipment according to embodiments of the present disclosure:
the thickness of the film is between 50 and 100 m;
the thickness of the film is between 5 and 10 times the nominal size of the transferable particles;
the surface area of the interaction zone is greater than 0.05 mm²;
the inlet leads to a lateral part of the interaction zone;
the interaction zone has a peripheral part opening laterally into the outlet;
the inlet and the outlet are constituted by tubular channels connected to the connection zone, the longitudinal axis of the tubular channels forming, with the transverse plane of the interaction zone, an angle between 15° and 350°;
the means for circulating the fluid between the inlet and the outlet include means for controlling the injection rate (or positive pressure exerted on the fluid) and the suction rate to control the flow of the fluid (or negative pressure exerted on the fluid) in the interaction zone;
the injection rate and suction rate control means are controlled by measuring the film thickness, to control the film thickness in the interaction zone;
the equipment has a plurality of interaction zones, each with an inlet and an outlet;
at least two of the interaction zones have common inlets and/or outlets;
the energy excitation means comprises a laser;
the interaction zone is transparent in the wavelength range of the laser and the imaging and does not have a sacrificial layer;
the fluid is charged with an absorbent pigment in the emission wavelength of the laser;
the equipment includes means for imaging the interaction zone for controlling the laser according to particle density;
the laser emits pulses in picosecond or femtosecond mode with an energy level between 5 and 20 microjoules, the energy level per pulse being controlled by a computer according to the result of the measurement of the characteristics of the fluid present in the interaction zone, the measurements including the particle density, and/or viscosity, and/or film thickness;
the energy excitation means consists of an acoustic wave generator; and
the equipment includes a means for imaging the interaction area and selecting the type of particle to be transferred.

The present disclosure also concerns an additive printing method by an equipment including a directable energy excitation means for producing a point interaction with a fluid covering a slide, in order to cause a jet oriented toward a target, the fluid being constituted by a liquid vector containing transferable inhomogeneities (particles, or biomaterials or chemical species) or by a transferable liquid biomaterial, characterized in that the fluid forms a liquid film of a thickness of less than 500 μm circulating between an inlet duct and an outlet duct of a slide having at least one zone allowing interaction with the laser, and into which at least one inlet opens.

According to a particular variant, the energy level per pulse is controlled by a computer according to the result of the measurement of the characteristics of the fluid present in the interaction zone, the measurements including particle density, and/or viscosity, and/or film thickness.

### BRIEF DESCRIPTION OF THE DRAWINGS

The present disclosure will be best understood upon reading the following detailed description of a non-limiting exemplary embodiment, while referring to the appended drawings, wherein:

FIG. 1 is a cross-sectional schematic view of an equipment according to the present disclosure;

FIG. 2 is a schematic view of an equipment according to the present disclosure with the optical system;

FIG. 3 is a schematic view from above of a slide for an equipment according to the present disclosure;

FIG. 4 is a 3D view of various variants of the equipment according to the present disclosure experimentally implemented;

FIG. 5 represents a 3D view of different variants of the equipment according to the present disclosure with a groove for imaging the ink film from the edge;

FIG. 6 shows a 3D view of a variant of the equipment according to the present disclosure as well as images of ink films obtained in this configuration;

FIG. 7 is a 3D view of a multi-cavity variant of the equipment according to the present disclosure;

FIG. 8 represents a series of explanatory diagrams of the laser-matter interaction process without sacrificial layer implemented in the equipment according to the present disclosure;

FIG. 9 represents a series of photographs showing the jet of matter generated by laser—matter interaction without sacrificial layer within the equipment according to the present disclosure;

FIG. 10 shows a micrograph of a field printed with water droplets within the equipment according to the present disclosure in static mode (without continuous refilling);

FIG. 11 shows a micrograph of a field printed with droplets containing microbeads within the equipment according to the present disclosure in static mode (without continuous refilling);

FIG. 12 shows a micrograph of a field printed with droplets containing cells within the equipment according to the present disclosure in static mode (without continuous refilling);

FIG. 13 shows a micrograph of a printed field of water droplets produced within the equipment according to the present disclosure in dynamic mode (with continuous refilling);

FIG. 14 shows a micrograph of a printed field of droplets containing cells as well as melanin (absorber) within the equipment according to the present disclosure in static mode (without continuous refilling); and

FIGS. 15 to 17 show views of other embodiments of a slide according to the present disclosure.

### DETAILED DESCRIPTION

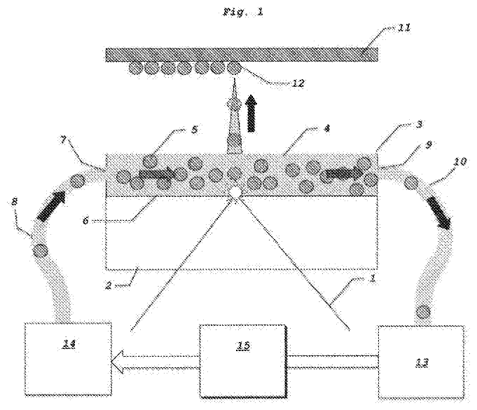

FIG. 1 shows a first variant embodiment of the equipment, using excitation by a focused laser beam generating a laser beam (1).

A slide (2) made of glass or transparent material defines a cavity (3) in which flows a carrier fluid (4) containing transferable particles (5).

The depth of this cavity is less than 500 m and preferably from 50 to 100 m thick, thus avoiding settling phenomena in the cavity (3).

This cavity (3) is formed by molding, machining, blowing (glass) or 3D printing (FDM, SLS, SLA, DLP, DMLS, EBM, CLIP, MultiJet, etc.) and has a circular, or rectangular, or oval, section, or other geometric shapes. Its transverse surface (6) defines a working area that can be scanned by the laser beam (1) and visualized on a sensor via an optical retrobeam.

The carrier fluid (4), pushed by a pumping system (15), enters the cavity (3) through an inlet (7) connected to a supply duct (8) itself connected to a supply tank (14), and is discharged via an outlet (9) to a discharge and/or exhaust duct (10).

The discharge and/or exhaust duct (10) leads to a recovery tank (13) containing the carrier fluid (4) loaded with transferable particles (5). A pumping system (15) circulates the carrier fluid (4) loaded with transferable particles (5). The supply tank (14) and the recovery tank (13) can be separated or form a single tank if the same fluid is to be recirculated in the system several times. In this configuration, the interest is to maximize the number of particles printed in the circulating fluid.

Optionally, the system includes several sets of supply tanks (14 and 13), each containing a carrier fluid loaded with inhomogeneities of different kinds. A valve is used to select one of the tanks, to allow the deposition of particles of different kinds and the formation of differentiated layers on the target (11).

The flow rate of the carrier fluid (4) is adjusted to ensure that the working area of the transferable particles (5) is moved at a speed that allows selection by appropriate means (imaging, spectroscopy, etc.) and activates those selected by a laser pulse.

The target (11) is movable in a plane X, Y parallel to the transverse surface (6) of the cavity (3) to determine the deposition point of the transferred particle (12) and optionally in a perpendicular direction, to adjust the distance travelled by the transferable particle (5) to be transferred. In this case, it is possible to modulate the size of the droplets deposited on the printing substrate.

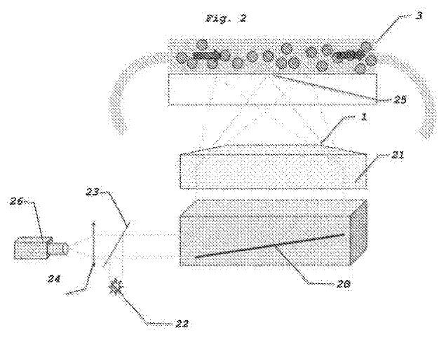

FIG. 2 is a cross-sectional view of the device combined with the optical system.

This optical system consists of two angularly oscillating mirrors (galvanometer type) (20), allowing to scan the laser shooting area, and a first optical unit (21) consisting of a scanning lens, of F-Theta type, allowing to form a laser spot whose diameter on the working plane is the smallest and most constant possible. This first optical unit (21) is made up in a known way by a system of several lenses.

Upstream of the scanning mirrors (20), the optical system includes a laser source (22) whose beam is reflected back to the scanning mirrors (20) by a dichroic mirror (23).

A second optical unit (24) forms an image of the working area (25) using the retro-beam passing through the dichroic mirror (23), on a sensor (26).

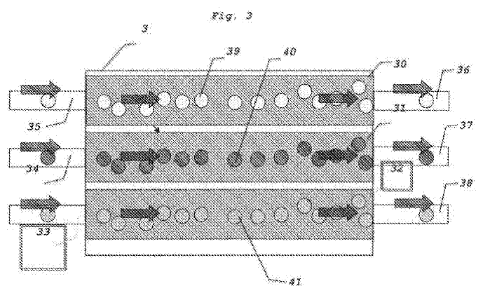

FIG. 3 is a top view of a variant of a slide (2) according to the present disclosure.

It consists of three circuits consisting of three parallel cavities (30, 31, 32), each extending between a supply duct (33 to 35 respectively) and an exhaust duct (36 to 38 respectively).

Each circuit ensures the circulation of a carrier fluid containing transferable inhomogeneities (39 to 41) of potentially different natures. Indeed, either they are of the same nature that could allow larger fields in size to be printed or the printing process to be accelerated (gain in productivity), or they are of a different nature that could make it possible to manufacture complex and customized items (gain on the range of manufacturable items), it is the “multicolor” aspect provided by this type of architecture.

To select one of the cavities, the slide (2) can be mechanically moved in a direction perpendicular to the main axis of the three cavities, or the scanning of the laser beam can be made to cover the entire slide (2).

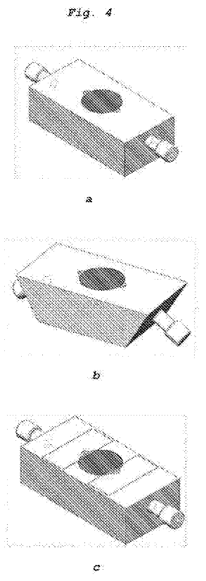

FIG. 4 shows a view of three possible architectures of the equipment. FIG. 4.a represents a view where the supply duct (8) and exhaust duct (10) are parallel to the slide (2) while FIG. 4.b illustrates a situation where the ducts arrive to the slide at an angle that can be between 0 and 90°. The interest of one or the other of the solutions lies in the ability to manage flows, dead volumes or angles in order to avoid any problem of clogging or continuity in the flow of the fluid (4) and to guarantee at the same time the obtaining of a homogeneous film of fluid in the open cavity (3) (circular part on the figures where the laser pulses are focused). FIG. 4.c is a variant of the previous solutions where glass lamellae have been placed at the inlets (7) and outlets (9) of the fluid (4) to direct/guide its flow as a homogeneous film in the cavity (3). The architectures illustrated here are not exhaustive. Indeed, the shape, positioning and angle of the ducts, the size and shape of the cavity and slide, the materials used, the shape and positioning of the inlet (7) and outlet openings may differ from the examples shown here.

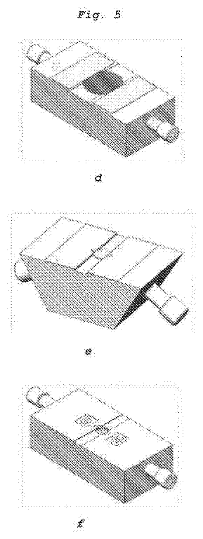

FIG. 5 shows a view of three possible equipment architectures similar to those shown in FIG. 4. They all have in common that in this new configuration they have a groove on the upper part whose height corresponds to that of the cavity (3) and whose positioning intersects the open area of the cavity (3). The advantage of such an opening is that it allows the observation of the film by visualization means (imaging) placed perpendicular to the equipment. Thus, it is possible to follow the evolution of the film over time, which allows adaptation of the energy of the laser pulses to the actual thickness of the film during printing or conversely adaptation of the thickness of the film to the energy of the laser pulse. Again, the architectures illustrated here are not exhaustive. Again, the shape, positioning and angle of the ducts, the size and shape of the cavity and slide, the materials used, the shape and positioning of the inlet (7) and outlet openings, the shape, size and positioning of the grooves may differ from the examples shown here.

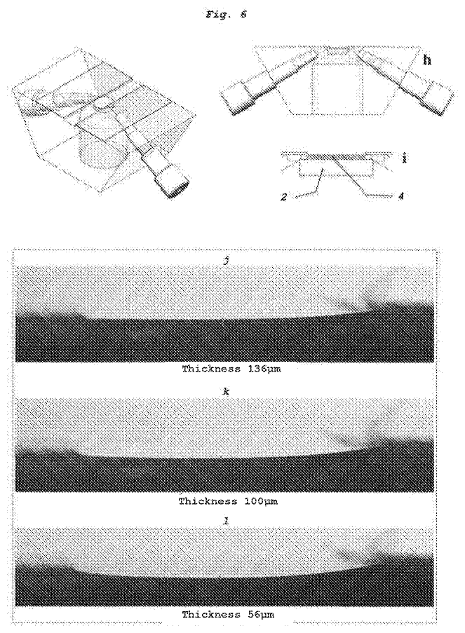

FIG. 6 shows results obtained on one of the variants of the equipment according to the present disclosure, initially illustrated in FIG. 5.e. In FIG. 6.h, a transparency view highlights the internal architecture of the equipment in this configuration. The supply and exhaust ducts come at an angle relative to the cavity (3) and the slide (2). The connection between the ducts and the cavity area is made by small diameter pipes to be compatible with the required film thicknesses of less than 500 μm. However, the cross-section of these ducts must not be too small in order to avoid any phenomenon comparable to those observed in nozzle systems (orifices) that clog easily and that bring significant mechanical stress to the cells, thus impacting their viability over time. For this reason, the ducts will preferably have a diameter greater than 200 μm, which is at least 10 times the average size of the cells or particles that can be printed by this equipment. It can be seen that the equipment is drilled in its center to form a hole (cylinder) allowing the laser to be focused in the cavity by passing through the slide (2), which is transparent to the wavelength of the laser. In FIG. 6.i, a zoomed view of the cavity (3) above the slide (2) to which the fluid (4) is sent shows that the inlet (7) and outlet (9) openings respectively are partly formed by the inlet and outlet ducts (8 and 10) respectively and by the glass lamellae placed on top of the equipment.

The combination of ducts and lamellae ensures that the fluid (4) is properly directed onto the slide (2). Images proving that thin films are possible are shown in photographs 6.j, 6.k and 6.l obtained by imaging (camera and image recovery lens) across the equipment. Depending on the pressure parameters of the fluid (4) at the inlet (7) and the suction of the fluid (4) at the outlet, it is possible to modulate the central thickness of the film. For example, thicknesses of 136 μm, 100 μm and 56 μm were obtained experimentally and are illustrated in FIGS. 6.j, 6.k and 6.l.

The monitoring of these thicknesses correlated to the adaptation of the laser excitation parameters (energy, focusing, etc.) allows the fine adjustment of the jets generated by the laser absorption. The upper shape of the film is not necessarily flat as can be seen in the photographs. This depends on the parameters of the fluid (4) (viscosity, density, flow rate . . . ) and the pressure/suction parameters of the fluid. The laser shooting area can be spatially adapted to a part of the film where the thickness is constant. This zone can also correspond to the entire cavity, but in this case, the laser parameters will be adapted to the variations in film thickness in the target field.

The characterization of the film could also be carried out by other means than the imaging mentioned here, such as spectroscopic analysis, distance measurement, line shadowgraph, etc.

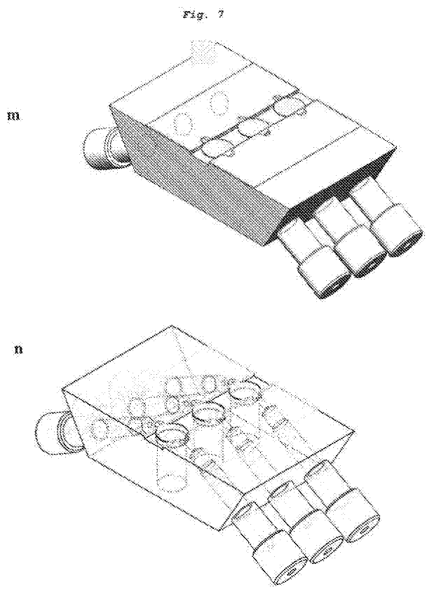

FIG. 7 is a 3D view of the solution shown in FIG. 3. This embodiment, shown in 3D in FIG. 7.m, has 3 cavities, each fed by dedicated supply and exhaust ducts. A groove is present on the top to allow the observation of the ink films, at least those placed at the ends of the equipment. In addition, a transparency view is provided in FIG. 7.n. It allows the three holes to be seen, allowing the laser to pass through the part to each cavity (3) equipped with a slide (2). It is obvious that this example is purely illustrative of the wide design possibilities that are possible for this equipment. Indeed, one could imagine it with:
two cavities or more than three cavities;
a single supply and/or exhaust duct common to all cavities;
a different cavity shape (square, canal, oval, diamond, etc.); and
ducts arriving parallel or perpendicular to the surface of the slides.

The examples cited here are therefore not limiting to the architectures that the equipment according to the present disclosure could have.

### Detailed Description of the Printing Method without Sacrificial Layer According to the Present Disclosure

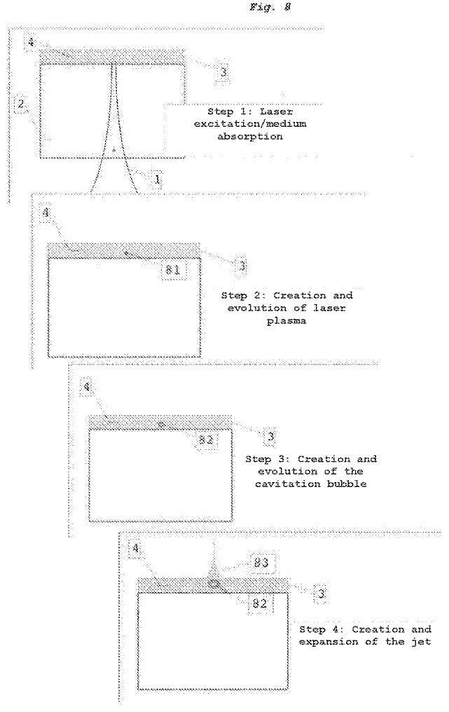

FIG. 8 describes the main steps of the interaction between laser and matter in the printing of homogeneous or inhomogeneities-containing liquids.

The first step involves focusing the laser on the material, which in this case is the ink arranged in the form of a film (4) in the cavity (3). The way the laser is focused directly impacts the volume that will absorb the deposited energy. This is called laser fluence (energy in relation to surface or volume). As the method does not use a sacrificial layer, it is the ink and most of its liquid medium that absorbs the laser energy. In fact, the choice of laser wavelength and energy has a direct impact on the absorption capacity of the film (4).

In the case of bio-printing, the ink is essentially composed of water that has well-known absorption peaks at the spectral level. It will therefore be possible to try to maximize this absorption by choosing laser sources corresponding to these maxima (e.g., infra-red water absorption lines). It may also be possible to try to maximize absorption through absorbers placed in the ink (molecules, dyes, particles). In the examples illustrated in the present disclosure, the laser used works at 1030 nm wavelength (ytterbium) for a pulse duration covering a range from 10 picoseconds to 400 femtoseconds and energies between 1 and 40 μJoules. Preferably, a laser with a pulse duration of 10 picoseconds for an energy per pulse of 10 to 14 μJoules was used.

The second step corresponds to the creation of the plasma (81), which is the result of the dissociation of the material following the absorption of the laser beam by the film fluid (4). This plasma is made up of a mixture of atoms, ions, electrons, molecular residues, etc.

The plasma is created over extremely short times, typically a few picoseconds after laser absorption and has a very short “life time” on the order of one microsecond. The size of the plasma (81), its spatio-temporal dynamics, its “temperature”, and its components are very strongly related to the duration of the laser pulse used. If the latter is in a so-called “short” regime from the microsecond to the nanosecond, the main effects at the origin of plasma are linear absorption effects with local temperature increases on the order of one to a few degrees. It is a “thermal” process. It is considered to be more “coarse” on the quality of plasma containment in a well-controlled and small space. On the other hand, if the pulse duration is in a so-called “ultra-short”regime, i.e., corresponding to pulse durations of a few tens of picoseconds to one femtosecond, then the effects at the origin of the plasma will be a combination of linear and non-linear effects.

Moreover, the shorter the pulse duration, the more non-linear effects will be favored. The advantage of using these regimes lies in the access to so-called “athermal” processes to ensure plasma containment in a very well bounded and very small space without temperature rise. This regime is therefore more favorable to cell viability a priori as well as to high resolution. In the case of the present disclosure, the main results were obtained between 5 and 10 picoseconds, a regime that mixes both linear and non-linear effects. They demonstrated the ability to print both homogeneous and colloidal media without sacrificial layers.

The third step is to create the cavitation bubble (82) in the medium. This bubble is the result of the recombination of the plasma components into a pressurized gas. Recombination is based on many complex physical processes such as field effects, radiative and non-radiative recombinations, tunnel effects, etc. . . . . Cavitation is very strongly dependent on the size and quality of the initial plasma (81). Cavitation bubble (82) appears after about one microsecond following absorption by the laser and the creation of the plasma. It can have a spherical shape but can also have an elongated or annular shape. It all depends on the initial plasma and its shape. The polarization of the laser and the geometric distribution of its energy at the focal plane have a direct influence on the shape of the plasma and therefore on the shape of the cavitation bubble. Thus, to obtain more reproducible results, isotropic forms, such as circular laser polarization, will be preferred.

Lastly, the fourth step corresponds to the so-called hydrodynamic phase where the cavitation bubble (82) will grow, deform, cause liquid movements, etc. The different phases of these hydrodynamic phenomena are already partly known through certain theories such as those of Pearson or Wortington, etc. The final result is the creation of a material jet (83) at the free surface of the liquid. The surface tension of the liquid, the distance from the bubble to the free surface, the viscosity of the liquid are among the most influential parameters on the shape and dynamics of this jet (83).

Thus, printing without a sacrificial layer will depend on a very large number of parameters related to both the laser and the ink used. The control of the ink film by the described equipment according to the present disclosure is a means of regulating some of the possible disparities (sedimentation, drying, variable and uncontrolled thickness, . . . ) during printing. In addition, the possibilities of modulating the flow rate and thickness of the film by means of pressure and suction could make it possible to modulate the size, shape and dynamics of the jets. Thus, with such a disclosure, it becomes possible to reduce the range of laser parameters required to modulate the jets. The direct impact of such a choice would be to use a laser that is much simpler in its definition, more stable and above all much less expensive because it is less versatile.

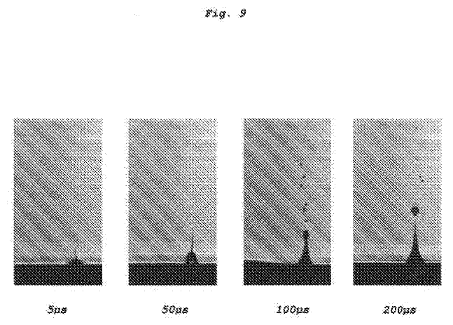

FIG. 9 illustrates actual jets of material, generated by laser without any sacrificial layer. The 4 photographs in this figure correspond each to a specific time after the laser pulse has been focused in the fluid (4). The first picture was taken 5 μs after the shooting, the second 50 μs after and so on. This shadowgraph imaging technique is commonly referred to as time-resolved imaging. It allows to break down hyper-fast events through photo shots with very short lighting times. This series of photos illustrates the principle of laser jet generation as explained in FIG. 8 above. Over short times, the creation of a pyramidal dome surmounted by a very fine first jet can be seen, then over longer times, the rise of a much more imposing jet from which one or more drops stand out can be seen. Depending on the distance between the free surface of the film (4) and the receiving substrate on which to print, one or more of these droplets will be deposited. Sometimes, it may happen that the distance between the ink and the printing substrate is small enough, usually less than 500 μm, for the jet to directly intercept the surface of the receiving substrate. This is referred to as a transfer regime. In all cases, whether the mechanism is droplet deposition or transfer, it will be called forward laser printing.

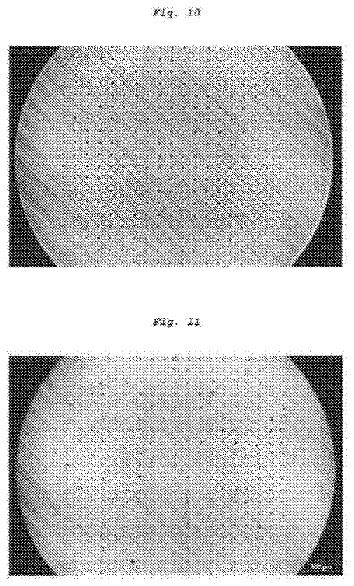

The following FIGS. 10, 11, 12, 13 and 14 illustrate the results obtained when printing without any sacrificial layer. These results prove that the parameter sets used in the present disclosure:
laser (picosecond regime, ten microJoules, near infra-red wavelength, polarization, etc.);
ink (viscosity, surface tension, density, thickness, etc.);
system (scanning speed, pattern used, focus, etc.); and
allow to print homogeneous as well as inhomogeneous items, which had never been demonstrated before.

Thus, FIG. 10 shows a highly reproducible laser printing result without any sacrificial layer of a homogeneous ink consisting mainly of water. Each printed drop appears as a small circle on the image. The large circle (separating the grey area from the black area) simply corresponds to the field imaged by the microscope used to take this photograph. The printed drops are typically 100 μm in diameter and 500 μm apart. This result was obtained within the equipment according to the present disclosure in static mode (without continuous refilling).

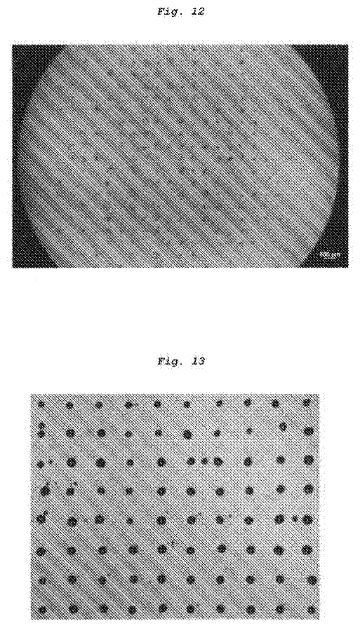

FIG. 11 shows a homogeneous result of laser printing without sacrificial layer of colloidal ink made of water, surfactant and microbeads of 5 μm diameter each. The printing result shows the ability of the present disclosure to deposit droplets containing a small number of microbeads, on average 2 to 3 per droplet. This is proof that printing without any sacrificial layer can achieve very high resolution performance on colloidal media, (which has never been demonstrated before). This result was obtained within the equipment according to the present disclosure in static mode (without continuous refilling).

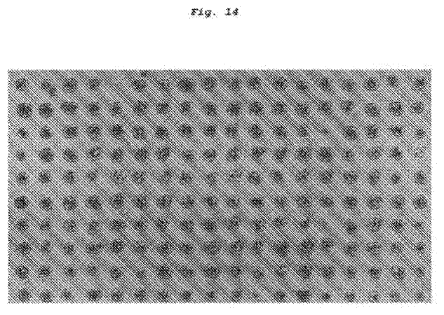

FIG. 12 shows a relatively homogeneous result of laser printing without any sacrificial layer of cellular ink. This printing is a first as for microbeads. This illustrates the very wide fields of use in which embodiments of the present disclosure may be employed. The printing disparities visible on the image are essentially related to the disparities of the ink used and deposited on the slide (2), which has settled and aggregated in clusters. Indeed, this result was obtained in static mode, i.e., without operating the equipment according to the present disclosure in a dynamic continuous refilling mode. The main purpose of this result was to prove the ability of gold-free printing to print living cells. Again, this result was obtained within the equipment according to the present disclosure in static mode (without continuous refilling).

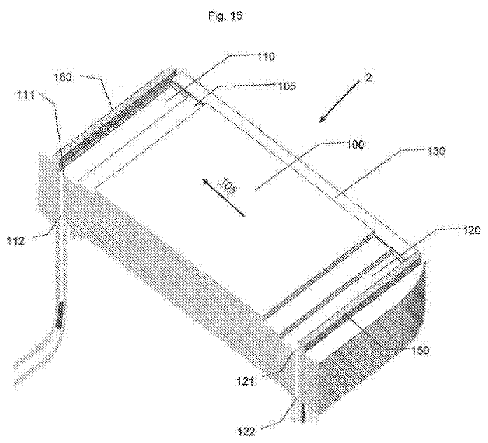

FIG. 13 shows a relatively homogeneous printing result of laser printing without sacrificial layer with a homogeneous ink obtained with the equipment according to the present disclosure working in dynamic mode, i.e., with the fluid system working in continuous refilling.

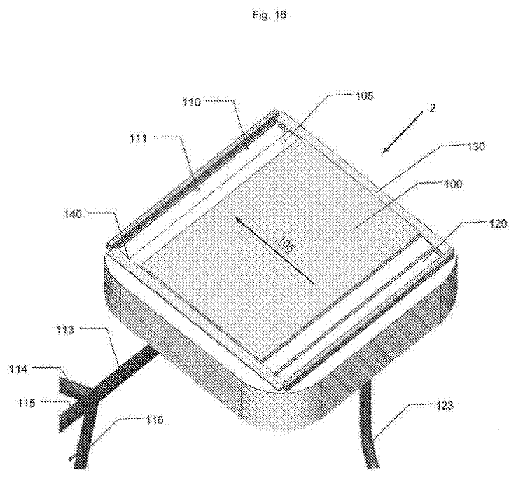

Lastly, FIG. 14 is another illustration of the ability of sacrificial-layer-less printing technology to be optimized according to needs. Indeed, in this image, a field of droplets printed under the same conditions as those described so far can be seen, with only one difference: the presence of an absorbent agent incorporated into the ink. In this example, it was melanin, a natural biological compound with a very high absorption at the wavelength of the laser used for these experiments, namely 1030 nm. Thus, the addition of this compound made it possible to work at lower laser energies to allow laser absorption, then plasma creation and finally the hydrodynamics of the cavitation bubble. This result was obtained within the equipment according to the present disclosure in static mode (without continuous refilling).

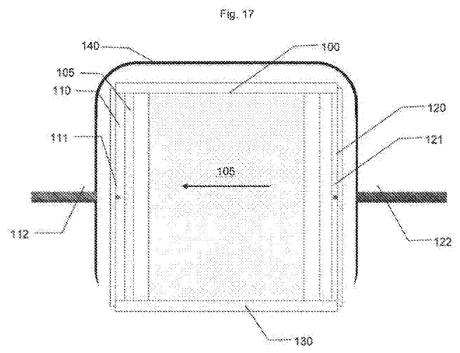

FIGS. 15 to 17 represent partial section, perspective and top views, respectively, of another example of a slide (2), with an optional first axis (105), and a mesa-shaped plate (100), with a flat upper surface. This flat surface defines the area of interaction between the fluid and an excitation and/or observation beam, for example, a laser beam.

The slide has a transverse groove (110, 120) on either side of this plate (100).

Each of the grooves (110, 120) communicates through a hole (111, 121) with a respective duct (112, 122) vertically passing through the slide (2) and opening into the corresponding groove respectively (110, 120).

The flow of the fluid occurs in a direction represented by the axis (105) corresponding to the longitudinal direction, between a first transverse groove (110) and a second transverse groove (120).

The first groove (110) is normally used for fluid supply, which passes through the plate (100) before flowing into the second groove (120) where the fluid is then sucked up. However, it is also possible to change the flow direction temporarily, so as to ensure an alternating flow at the surface of the plate (100).

Tubes (113, 123) are connected respectively to the hole (111, 121) for the supply and/or suction of fluid carrying the transferable particles. One of the ducts can be connected by a multi-way valve to several inlets (114 to 116) of fluids of different types. Each of these channels can work either in flow rate (such as syringe pump) or pressure

The other two edges of the plate (100), not adjacent to the grooves (110, 120), are optionally bordered by a flange (130, 140) to form a delimited fluid flow area. Similarly, the outer lateral edges of the slide are delimited by walls (150, 160).

The control of the supply and/or suction flow rate makes it possible to control the flow to ensure a homogeneous distribution of the liquid over the open part of the print head.

A first solution is to provide an inlet port and a rectangular outlet, the inlet and outlet ports being defined by the upper surface of the transverse grooves (110, 120) as shown in FIGS. 15 to 17.

The lateral edges (130, 140) ensure that the shape of the liquid meniscus in the plane containing the axis (105) can be controlled. They can be physical or chemical.

According to a particular embodiment of the present disclosure, the liquid film has a perfectly flat or thinner thickness at the edges so that the flow has a higher hydrodynamic resistance at the edges.

This solution may also include:
different elements to be able to control the thickness of the liquid;
walls (150, 160) upstream and downstream on which inking of the contact lines is facilitated; and
a central plate (100) for raising the film to reduce the thickness of the film in the interaction area.

Another solution to alternate the nature of the fluid is to alternate between a fluid containing transferable particles and an inert liquid.

Another solution consists of a liquid discharge pipe with at least two channels.

The operation is then as follows:
Step 1 of initialization:
   In order to create a liquid bridge on the print head connecting the injection point and the discharge point, a control process is set up.
   A first solution is to inject liquid from the cartridges to the head from the injection and discharge point.
   Another solution is to inject liquid from the cartridge to the head only from the injection point.
   Another solution is to use a texture that allows the open part of the print head to be completely wetted.
Automation step 2:
   Once the liquid bridge is created, the film thickness is controlled by removing liquid either from the injection point or from the discharge point.
   A first example of embodiment establishes a continuous flow and controls the thickness of the film by imposing a flow rate between the injection point and the discharge point.
   Another example of embodiment establishes a continuous flow and controls the thickness of the film by imposing a pressure difference between the injection point and the discharge point.
   Another example of embodiment operates in batch mode:
      injection of an imposed volume from the injection point; and
      then adjusting the desired thickness by removing liquid from the injection or discharge point.

This operation is repeated cyclically.

In order to better control the liquid film on the print head, sensors can be integrated.

An example of embodiment uses a confocal system to measure the thickness of the film. Another example of embodiment uses an optical detection system on the injection and discharge ducts. An advantageous development makes it possible to detect the passage of bubbles, particularly on the escape route. An advantageous development makes it possible to detect concentrations, particularly on the injection route.

1. An additive printing apparatus, comprising:
a slide having at least one zone allowing interaction between a fluid film on the slide and an energy beam, a fluid inlet allowing fluid flow into the at least one zone, and a fluid outlet allowing fluid to flow out from the at least one zone;
an orientable energy excitation device for producing a point of interaction between an energy beam emitted by the energy excitation device and a fluid film on the slide to cause a jet oriented toward a target, the fluid comprising a liquid containing transferable inhomogeneities; and
a fluid circulation system configured to circulate fluid between the inlet and the outlet, wherein the slide and the fluid circulation system are configured to form a fluid film having a thickness of less than 500 μm on the at least one zone of the slide.

2. The apparatus of claim 1, wherein the slide and the fluid circulation system are configured to form a fluid film having a thickness of between 20 and 100 μm.

3. The apparatus of claim 1, wherein the slide and the fluid circulation system are configured to form a fluid film having a thickness that is between 3 and 10 times a nominal size of the transferable inhomogeneities.

4. The apparatus of claim 1, wherein the at least one zone of the slide has an area greater than 0.05 mm².

5. The apparatus of claim 1, wherein the fluid inlet opens into a lateral part of the at least one zone of the slide.

6. The apparatus of claim 1, wherein the at least one zone of the slide-has a peripheral part opening laterally into the fluid outlet.

7. The apparatus of claim 1, wherein the fluid inlet and the fluid outlet comprise tubular channels.

8. The apparatus of claim 1, wherein the fluid circulation system includes a controller for controlling a flow rate of the fluid of the fluid film on the at least one zone of the slide.

9. The apparatus of claim 8, wherein the fluid circulation system further includes a sensor for measuring the thickness of the fluid film, and where in the controller is configured to control the thickness of the fluid film in the at least one zone of the slide.

10. The apparatus of claim 1, wherein the at least one zone of the slide comprises a plurality of zones, each zone of the plurality further comprising a fluid inlet and a fluid outlet.

11. The apparatus of claim 10, wherein at least two zones of the plurality of zones have common fluid inlets and/or fluid outlets.

12. The apparatus of claim 1, wherein the energy excitation device comprises a laser.

13. The apparatus of claim 12, wherein the at least one zone of the slide is transparent to wavelengths of the laser, and does not have any sacrificial layer.

14. The apparatus of claim 13, wherein the fluid of the fluid film comprises an absorbent pigment in an emission wavelength of the laser.

15. The apparatus of claim 12, further comprising an imaging system configured to acquire images of the at least one zone of the slide.

16. The apparatus of claim 12, wherein the laser emits pulses in picosecond or femtosecond mode with an energy level between 20 and 40 microjoules, the energy level per pulse being controlled by a computer according to a result of a measurement of fluid characteristics present in the at least one zone of the slide, the measurements including density in inhomogeneities, and/or viscosity, and/or film thickness.

17. The apparatus of claim 12, wherein the laser emits pulses in nanosecond mode with an energy of 0.5 to 20 millijoules, the energy level per pulse being controlled by a computer according to a result of a measurement of fluid characteristics present in the interaction zone, the measurements including inhomogeneity density, and/or viscosity, and/or film thickness.

18. The apparatus of claim 1, wherein the energy excitation device comprises an acoustic wave generator.

19. The apparatus of claim 1, wherein the energy excitation comprises a surface wave generator configured to generate vibrations.

20. The apparatus of claim 1, further comprising an imaging system configured to acquire images of the at least one zone of the slide.

21. The apparatus of claim 1, wherein the slide comprises a mesa-shaped plate, an upper surface of which defines the at least one zone of the slide, the slide having on either side of the plate a transverse groove, each of the grooves communicating through a hole with a duct respectively vertically traversing the slide and opening into the corresponding groove respectively.

22. (canceled)

23. (canceled)

24. A method of additive printing, comprising:
providing a slide having at least one zone allowing interaction between a fluid film on the slide and an energy beam, a fluid inlet allowing fluid flow into the at least one zone, and a fluid outlet allowing fluid to flow out from the at least one zone;
providing an orientable energy excitation device for producing a point of interaction between an energy beam emitted by the energy excitation device and a fluid film on the slide in the at least one zone to cause a jet oriented toward a target, the fluid comprising a liquid containing transferable inhomogeneities; and
circulating fluid between the inlet and the outlet of the slide and forming the fluid film on the slide, the fluid film having a thickness of less than 500 μm on the at least one zone of the slide.

25. The method of claim 24, wherein the energy excitation device comprises a laser configured to generate a pulsed laser beam, the method further comprising:
measuring characteristics of the fluid film in the at least one zone of the slide, the measurements including particle density, and/or viscosity, and/or film thickness; and
controlling an energy level per pulse of the pulsed laser beam using a computer as a function of the measured characteristics of the fluid film in the at least one zone of the slide.
